(19) United States
(12) Patent Application Publication
(10) Pub. No.: US 2014/0046669 A1
(43) Pub. Date: Feb. 13, 2014

(54) SYSTEM AND METHOD FOR BIOMEDICAL MEASUREMENT WITH VOICE NOTIFICATION FEATURE

(71) Applicant: HEALTH & LIFE CO., LTD., New Taipei City (TW)

(72) Inventor: Meng-Yi LIN, New Taipei City (TW)

(73) Assignee: HEALTH & LIFE CO., LTD., New Taipei City (TW)

(21) Appl. No.: 13/900,667

(22) Filed: May 23, 2013

(30) Foreign Application Priority Data
Aug. 7, 2012 (TW) 101128419

(51) Int. Cl. A61B 5/00

(52) U.S. Cl. CPC A61B 5/741
USPC 704/274

(57) ABSTRACT

A system for biomedical measurement with a voice notification feature includes a biomedical measurement device that generates a command based on at least one of a physiological signal obtained by the biomedical measurement device as a result of a measured physiological condition of a user, and an operation phase of the biomedical measurement device, and a mobile device that is coupled to and separate from the biomedical measurement device. The mobile device receives the command from the biomedical measurement device, generates a voice notification signal according to the command and pre-established voice data, and outputs audibly the voice notification signal.

### CROSS-REFERENCE TO RELATED APPLICATION

This application claims priority to Taiwanese Application No. 101128419, filed on Aug. 7, 2012.

### BACKGROUND OF THE INVENTION

1. Field of the Invention

The invention relates to a system and a method related to biomedical measurement, and more particularly to a system and a method for biomedical measurement with a voice notification feature.

2. Description of the Related Art

Recently, handhold biomedical measurement devices have been commonly used in general living for measuring personal physiological conditions, such as blood pressure, heart rate, blood sugar, etc.

For portability, the biomedical measurement devices are usually made to be thin and light, so that a display thereof does not have a sufficient size, resulting in difficulty of operation for an elder or a user without normal visual acuity.

In order to solve this problem, it has been proposed to add voice functionality to the biomedical measurement devices for guiding user operation, and relevant voice data is stored in the biomedical measurement devices. For satisfying the global market, various language versions of the voice data must be stored therein, resulting in requirement of large storage space and high cost. However, a user usually employs only one language and the other language versions of the voice data are unnecessary for the single user and result in unwanted expense.

### SUMMARY OF THE INVENTION

Therefore, an object of the present invention is to provide a system for biomedical measurement with a voice notification feature.

According to one aspect of the present invention, a system for biomedical measurement with a voice notification feature comprises:

a biomedical measurement device configured to generate a command based on at least one of a physiological signal obtained by the biomedical measurement device as a result of a measured physiological condition of a user, and an operation phase of the biomedical measurement device; and

a mobile device that is coupled to and separate from the biomedical measurement device, the mobile device being configured to receive the command from the biomedical measurement device, to generate a voice notification signal according to the command and pre-established voice data, and to output audibly the voice notification signal.

Another object of the present invention is to provide a mobile device for implementing the voice notification feature of the system of this invention for biological measurement.

According to another aspect of the present invention, a mobile device is adapted for use with a biomedical measurement device that is configured to generate a command based on at least one of a physiological signal obtained by the biomedical measurement device as a result of a measured physiological condition of a user, and an operation phase of the biomedical measurement device. The mobile device is separate from the biomedical measurement device and comprises:

a transceiver module for coupling to the biomedical measurement device so as to receive the command therefrom;

a voice processing module for generating a voice notification signal according to the command received by the transceiver module and pre-established voice data; and

an output module for outputting audibly the voice notification signal.

Yet another object of the present invention is to provide a method for performing the voice notification feature of the system for biomedical measurement of this invention.

According to yet another aspect of the present invention, a method for performing the voice notification feature of the system for biomedical measurement of this invention is implemented by a mobile device that is coupled to and separate from a biomedical measurement device. The method comprises:

a) configuring the mobile device to receive a command from the biomedical measurement device, the command being generated by the biomedical measurement device based on at least one of a physiological signal obtained by the biomedical measurement device as a result of a measured physiological condition of a user, and an operation phase of the biomedical measurement device;

b) configuring the mobile device to generate a voice notification signal according to the command received in step a) and pre-established voice data; and

c) configuring the mobile device to output audibly the voice notification signal generated in step b).

### BRIEF DESCRIPTION OF THE DRAWINGS

Other features and advantages of the present invention will become apparent in the following detailed description of the preferred embodiment with reference to the accompanying drawings, of which:

FIG. 1 is a schematic diagram illustrating an application of the system for biomedical measurement with a voice notification feature according to the present invention;

FIG. 2 is a block diagram illustrating a preferred embodiment of the system for biomedical measurement with a voice notification feature according to the present invention; and

FIG. 3 is a flow chart illustrating steps of the method for performing the voice notification feature of the system for biomedical measurement of this invention.

### DETAILED DESCRIPTION OF THE PREFERRED EMBODIMENT

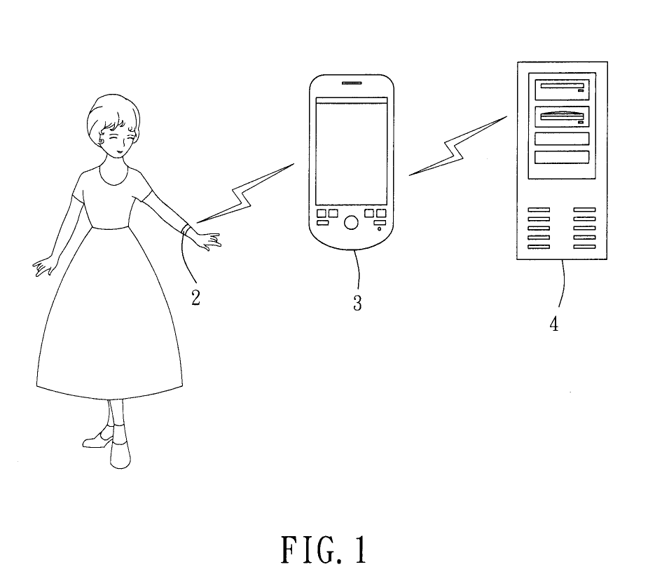

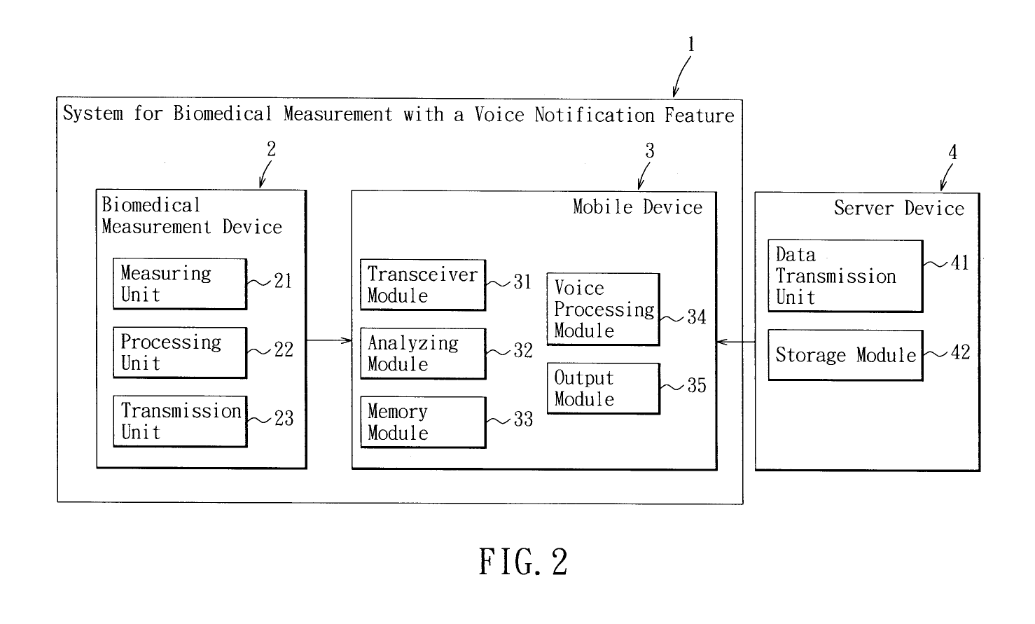

Referring to FIG. 1 and FIG. 2, the preferred embodiment of the system 1 for biomedical measurement with a voice notification feature according to this invention is shown to include a biomedical measurement device 2 and a mobile device 3 separate from the biomedical measurement device 2. The biomedical measurement device 2 includes a measuring unit 21, a processing unit 22, and a transmission unit 23. The mobile device 3 includes a transceiver module 31, an analyzing module 32, a memory module 33, a voice processing module 34, and an output module 35.

In the preferred embodiment, the biomedical measurement device 2 is configured to be similar to the common biomedical measurement device, such as a blood pressure detector, a blood sugar detector, etc., and the mobile device 3 may be a smartphone.

It should be noted that, in this embodiment, the analyzing module 32 and the voice processing module 34 are implemented via execution of proprietary software by the mobile device 3. In practice, the software is an application product including an analyzing program and a voice processing program that are executable by the mobile device 3. After a processor (not shown) of the mobile device 3 loads and executes the application, the analyzing module 32 and the voice processing module 34 may be configured in the mobile device 3.

In this embodiment, the system 1 for biomedical measurement is required to obtain data for the voice notification feature from a server device 4 when the system 1 is used for the first time or needs to be updated. The server device 4 includes a storage module 42 that stores pre-established voice data, and a data transmission unit 41 for transmitting the pre-established voice data to the mobile device 3. It should be noted that the storage module 42 may be a hard disk drive or a flash memory, and the data transmission unit 41 may transmit the pre-established voice data to the transceiver module 31 of the mobile device 3. The pre-established voice data includes various different language versions of voice data packets, that is, voice files of vocabularies/phrases and syntax information (grammar). The pre-established voice data is associated with operation instructions of the biomedical measurement device 2.

The measuring unit 21 of the biomedical measurement device 2 is configured to obtain a physiological signal as a result of a measured physiological condition of a user. The physiological condition may be a blood pressure value or a blood sugar concentration value of the user. The processing unit 22 generates a command based on the results of physiological signal or the operation phases of the user, and the transmission unit 23 transmits the command to the mobile device 3.

In this embodiment, the transmission unit 23 may transmit the command to the mobile device 3 using a physical connection, such as a universal asynchronous receiver transmitter (UART), a universal serial bus (USB), etc., or using a radio frequency technique, such as Bluetooth, radio frequency identification (RFID), near field communication (NFC), Wi-Fi, etc. Details of transmission should be readily appreciated by persons having ordinary skill in the art, and thus are not described herein.

The transceiver module 31 of the mobile device 3 is adapted for coupling to the biomedical measurement device 2 so as to receive the command therefrom, and for coupling to the server device 4 so as to obtain the pre-established voice data therefrom. The analyzing module 32 is adapted for analyzing the command. The memory module 33 is adapted for storing the command and other data, such as the pre-established voice data. The voice processing module 34 is adapted for constructing a notification syntax based on a result obtained from analysis of the command and corresponding grammar rules included in the pre-established voice data, and for generating a voice notification signal according to the notification syntax. The output module 35 is adapted for outputting audibly the voice notification signal. In this embodiment, the memory module 33 may be a hard disk drive or a flash memory, and the output module 35 may be a speaker or a headset jack with an audio amplifier.

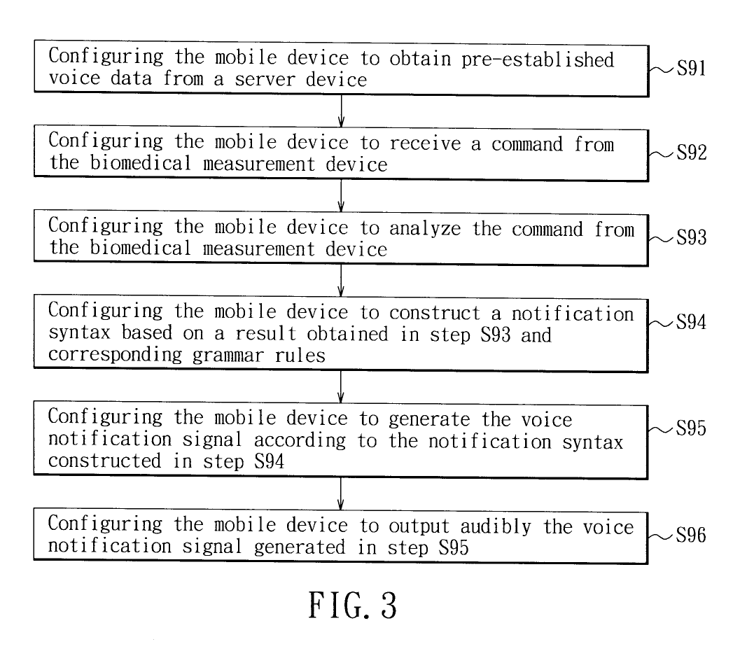

Referring to FIG. 2 and FIG. 3, the method for performing the voice notification feature of the system for biomedical measurement of this invention is implemented by a mobile device 3 that is coupled to a separate biomedical measurement device 2 and that is installed with an application program for execution of this method. The steps of the method of this invention are illustrated as follows.

Step S91: The transceiver module 31 of the mobile device 3 is configured to obtain the pre-established voice data from a server device 4, and to store the pre-established voice data into the memory module 33 when the application is used for the first time or the voice data stored in the memory module 33 is required to be updated.

It should be noted that, since the pre-established voice data of the storage module 42 of the server device 4 includes different language versions, such as Chinese, English, Japanese, French, etc., the transceiver module 31 may download only one of the language versions of the pre-established voice data from the server 4 according to a language setting of the mobile device 3 or a language option selected by the user via a user interface of the mobile device 3. For example, when the selected language option is “English”, the transceiver module 31 may download the English voice files of vocabularies/phrases, such as “blood sugar”, “concentration”, “higher”, “standard”, and corresponding grammar rules.

Voice data transmission between the transceiver module 31 of the mobile device 3 and the data transmission unit 41 of the server device 4 may be implemented using a physical connection or RF techniques, such as UART, USB, bluetooth, RFID, NFC, Wi-Fi, etc.

According to step S91, since the pre-established voice data is downloaded from the server device 4, the biomedical measurement device 2 is not required to include any storage device for storing the pre-established voice data. The memory module 33 of the mobile device 3 is only required to have sufficient space to store the downloaded pre-established voice data. Compared to the conventional biomedical measurement product, the present invention may save cost by omitting a storage device that is adapted for storing various language versions of the voice data.

Step S92: The transceiver module 31 of the mobile device 3 is configured to receive a command from the biomedical measurement device 2 using a physical connection or RF techniques. The command is generated prior to step S92 by the processing unit 22 of the biomedical measurement device 2 based on at least one of a physiological signal obtained by the biomedical measurement device as a result of a measured physiological condition of a user, and an operation phase of the biomedical measurement device. For example, when the measuring unit 21 measures concentration of blood sugar of the user, the processing unit 22 generates the command corresponding to the measured concentration of blood sugar for transmission by the transmission unit 23 to the mobile device 3.

Step S93: The analyzing module 32 of the mobile device 3 is configured to analyze the command from the biomedical measurement device 2. For example, assuming the analysis result obtained by the analyzing module 32 indicates the blood sugar concentration is higher than a standard, the analyzing module 32 outputs instructions corresponding to “blood sugar”, “concentration”, “high”, “standard” to the voice processing module 34.

Step S94: The voice processing module 34 of the mobile device 3 is configured to construct a notification syntax based on a result obtained in step S93 and corresponding grammar rules included in the pre-established voice data. Following the example provided in Step S93, since the instructions received from the analyzing module 32 represent physiological conditions of “the blood sugar concentration is higher than the standard”, the voice processing module 34 is configured to search from the memory module 33 for the related vocabularies, phrases, and grammar rules, that is, voice files and syntax information (grammar rules) that are associated with the “blood sugar concentration is higher than a standard”.

Step S95: The voice processing module 34 of the mobile device 3 is configured to generate the voice notification signal according to the notification syntax constructed in step S94. In other embodiments, the mobile device 3 may be configured to download the pre-established voice information from the server device 4 through the transceiver module 31 thereof in real-time for the voice processing module 34 to generate the voice notification signal. Details of generation of the voice notification signal should be readily appreciated by persons having ordinary skill in the art, and thus are not described herein.

Step S96: The output module 35 of the mobile device 3 is configured to output audibly the voice notification signal generated in step S95. The voice notification signal outputted by the output module 35 may be used for guiding operation of the biomedical measurement device 2 or notifying information associated with the measured physiological condition of the user.

To sum up, according to this invention, since the pre-established voice data is not stored in the biomedical measurement device 2, it is not required to provide a storage device therein for storing the pre-established voice data, thereby reducing cost and complexity of design. The voice notification signal is generated based on analysis of the command and the required voice data and is audibly outputted by the mobile device 3, the required storage space is reduced for the user, and the voice chip, speaker, and software program for voice output are not required for the biomedical measurement device 2.

While the present invention has been described in connection with what is considered the most practical and preferred embodiment, it is understood that this invention is not limited to the disclosed embodiment but is intended to cover various arrangements included within the spirit and scope of the broadest interpretation so as to encompass all such modifications and equivalent arrangements.

What is claimed is:

1. A system for biomedical measurement with a voice notification feature, comprising:
a biomedical measurement device configured to generate a command based on at least one of a physiological signal obtained by said biomedical measurement device as a result of a measured physiological condition of a user, and an operation phase of said biomedical measurement device; and
a mobile device that is coupled to and separate from said biomedical measurement device, said mobile device being configured to receive the command from said biomedical measurement device, to generate a voice notification signal according to the command and pre-established voice data, and to output audibly the voice notification signal.

2. The system as claimed in claim 1, wherein said mobile device is further configured to obtain the pre-established voice data from a server device.

3. The system as claimed in claim 2, wherein said mobile device is configured to obtain the pre-established voice data from the server device using one of radio frequency techniques and a physical connection.

4. The system as claimed in claim 1, wherein said mobile device is configured to analyze the command received from said biomedical measurement device, to construct a notification syntax based on a result obtained from analysis of the command and corresponding grammar rules included in the pre-established voice data, and to generate the voice notification signal according to the notification syntax.

5. The system as claimed in claim 1, wherein said mobile device is configured to obtain the command from said biomedical measurement device using one of radio frequency techniques and a physical connection.

6. A mobile device adapted for use with a biomedical measurement device that is configured to receive a command generated by the biomedical measurement device based on at least one of a physiological signal obtained by the biomedical measurement device as a result of a measured physiological condition of a user, and an operation phase of the biomedical measurement device, said mobile device being separate from the biomedical measurement device and comprising:
a transceiver module for coupling to the biomedical measurement device so as to receive the command therefrom;
a voice processing module for generating a voice notification signal according to the command received by said transceiver module and pre-established voice data; and
an output module for outputting audibly the voice notification signal.

7. The mobile device as claimed in claim 6, wherein said transceiver module is further configured to obtain the pre-established voice data from a server device.

8. The mobile device as claimed in claim 7, wherein said transceiver module is configured to obtain the pre-established voice data from the server device using one of radio frequency techniques and a physical connection.

9. The mobile device as claimed in claim 6, wherein said voice processing module is configured to analyze the command received by said transceiver module, to construct a notification syntax based on a result obtained from analysis of the command and corresponding grammar rules included in the pre-established voice data, and to generate the voice notification signal according to the notification syntax.

10. The mobile device as claimed in claim 6, wherein said transceiver module is configured to obtain the command from the biomedical measurement device using one of radio frequency techniques and a physical connection.

11. A method for performing the voice notification feature of the system as claimed in claim 1, said method to be implemented by the mobile device of the system and comprising:
a) configuring the mobile device to receive a command from the biomedical measurement device of the system, the command being generated by the biomedical measurement device based on at least one of a physiological signal obtained by the biomedical measurement device as a result of a measured physiological condition of a user, and an operation phase of the biomedical measurement device;
b) configuring the mobile device to generate a voice notification signal according to the command received in step a) and pre-established voice data; and
c) configuring the mobile device to output audibly the voice notification signal generated in step b).

12. The method as claimed in claim 11, further comprising, prior to step b), configuring the mobile device to obtain the pre-established voice data from a server device.

13. The method as claimed in claim 12, wherein the mobile device is configured to obtain the pre-established voice data from the server device using one of radio frequency techniques and a physical connection.

14. The method as claimed in claim 11, wherein step b) includes:
b-1) configuring the mobile device to analyze the command received in step a);
b-2) configuring the mobile device to construct a notification syntax based on a result obtained in sub-step b-1) and corresponding grammar rules included in the pre-established voice data; and
b-3) configuring the mobile device to generate the voice notification signal according to the notification syntax constructed in sub-step b-2).
